(19) United States
(12) Patent Application Publication
(10) Pub. No.: US 2010/0055756 A1
(43) Pub. Date: Mar. 4, 2010

(54) System and method for modifying biological cells using an ultra-short pulsed laser

(76) Inventor: Gregory Spooner, San Francisco, CA (US)

Correspondence Address: CARR & FERRELL LLP, 2200 GENG ROAD, PALO ALTO, CA, 94303, US

(21) Appl. No.: 12/231,245

(22) Filed: Aug. 28, 2008

(51) Int. Cl. C12N 13/00; C12M 1/00

(52) U.S. Cl. 435/173.4; 435/285.1

(57) ABSTRACT

A system and method for modifying a biological cell are presented. A beam of ultra-short pulses is generated. The beam is delivered to a mixture that includes a biological cell and a medium. The beam is focused to form a focal zone. The focal zone may be proximate to the biological cell. An event is generated at the focal zone that effectuates a modification to the biological cell.

### BACKGROUND

1. Technical Field

The present invention generally relates to ultra-short pulsed lasers. More specifically, the present invention relates to modifying biological cells using an ultra-short pulsed laser.

2. Description of Related Art

Biological cells are the basic structural unit of all living organisms. A biological cell is a microscopic structure containing nuclear and cytoplasmic material enclosed by a membrane. In the biological cells of animals, the membrane is pliable. The membranes of the biological cells of plants, as well as some algae, bacteria, and fungi, is rigid and may be referred to as a cell wall. The membranes of both plant and animal biological cells act, in part, as a filter permitting passage of small molecules and small proteins into the biological cell.

Oftentimes, it may be desirable to introduce foreign objects or substances into the biological cell in various applications. For example, the foreign objects may include genes, DNA, RNA, or any other molecule that contains genetic instructions (e.g., those used in development and functions of any living organism). The foreign objects may be introduced into the biological cell in an effort to alter certain characteristics of the biological cell. The substances may include, for example, drugs, medicines, or any chemical that, when introduced into the biological cell, alters a normal function of the biological cell. In some instances, the substance may be used in the treatment, cure, prevention, and/or diagnosis of a disease, or may be used to otherwise enhance physical or mental well-being. However, a challenge is presented due to restriction by the membrane of relatively large objects, such as DNA molecules, from entering the biological cell.

In gene therapy, for example, foreign genes may be introduced into the biological cell with an intention that the biological cell may express certain traits or characteristics of the foreign genes. A process of introducing the foreign genes to the biological cell may be known as genetic transfer. Genetic transfer may be achieved at least by one of two broad approaches, one involving biological vectors and the other entailing chemical or physical techniques. In the former approach, the vectors (i.e., any agent that acts as a carrier or transporter) are commonly viruses, such as retroviruses and adenoviruses. The viruses may introduce the foreign genes into the biological cell by what may be known as infection.

In the latter approach for gene transfer, commonly referred to as transfection, non-viral gene transfer is accomplished by chemical-based or physical-based methods. As one skilled in the art will recognize, transfection may generally refer to introduction of any material into the biological cells using any means of transfer. Chemical methods include use of an array of chemical complexes between DNA and polyplexes or lipoplexes to introduce the foreign genes into the biological cell. Generally, chemical methods may be readily scaled, but may suffer from poor efficiency and minimal targeting. Physical methods include mechanical transfection (e.g., microinjection and particle bombardment, also known as use of a “gene gun”), physical transfection (e.g., electroporation, also known as electropermeabilization, sonoporation, and optoporation), and magnetic field-enhanced transfection. Physical methods may present a potential to achieve rapid expression of the foreign genes by direct transference of the foreign genes into the biological cell.

Mechanical transfection includes physical methods where the foreign genes are driven into the biological cell by external force. In microinjection, each biological cell may receive the foreign genes by injection using, for example, a microscopic syringe. However, since microinjection is a serial approach (i.e., only one biological cell may be injected at a time), it is impractical for many applications. Particle bombardment involves impacting the biological cell with a transfection agent, such as a heavy metal particle that has been coated with the foreign genes. The intent of particle bombardment may be that the transfection agent will penetrate the membrane of the biological cell and the foreign genes will be released. Standard techniques for particle bombardment involve accelerating the transfection agent using, for example, a high-voltage electric spark or a helium discharge. Particle bombardment using the standard techniques may not generally be conducive to in vivo applications.

Physical transfection includes methods for altering a permeability of the membrane of the biological cell such that adjacent foreign genes may be absorbed into the biological cell. In electroporation, the biological cell may be briefly exposed to an electric field. During exposure, the permeability of the membrane of the biological cell to nearby foreign genes may increase. The apparatus for electroporation generally includes an electric pulse generator and electrodes. A drawback of electroporation may be that expression of the foreign genes may not be homogeneously distributed, for example, due to geometry of the electrodes. Ultrasound may be typically applied to the biological cell during sonoporation, such as by an ultrasonic bath or a sonography inducer, to temporarily increase the permeability of the membrane of the biological cell. Efficacy of, and toxicity resulting from, sonoporation involving, for example, the ultrasonic bath or the sonography transducer, may be inconsistent. Optoporation relies on laser irradiation and involves focusing a laser beam onto a surface of the membrane. The permeability may be changed at the site on the membrane that is impinged on by the laser beam, for example, by local thermal effects. The local thermal effects may permanently damage the membrane and may be undesirable in various applications, such as in vivo applications.

Magnetic field-enhanced transfection, or magnetofection, is a recently developed method where the foreign genes are associated with magnetic nanoparticles. An external magnetic field may be used to preferentially concentrate the magnetic nanoparticles near the biological cell leading to statistically increased transfection rates. Magnetofection is generally regarded as a method of enhancing other transfection methods that involve non-biological vectors rather than a standalone method.

### SUMMARY OF THE INVENTION

An exemplary system and method for modifying a biological cell are presented. A beam of ultra-short pulses is generated. The beam of ultra-short pulses may be generated, for example, by an ultra-short pulsed laser. The beam is then delivered to a mixture that includes a biological cell and a medium. In some examples, the beam may be coupled to an optical fiber that delivers the beam to the mixture. In other examples, the beam may be directed by conventional optical elements. The beam is focused to form a focal zone that is near the biological cell.

One or more events may be generated at the focal zone that brings about a modification to the biological cell. Each of the events may induce certain modifications to the biological cell. In some examples, the modifications may result in transfection of the biological cell. In other examples, the modification may affect permeability and/or porosity of a membrane of the biological cell.

### BRIEF DESCRIPTION OF THE DRAWINGS

FIG. 1 illustrates an exemplary system for modifying the biological cell.

FIG. 2 illustrates the focal zone, according to exemplary embodiments.

FIGS. 3A-3C illustrate an exemplary biolistic process for transfecting the biological cell using the system.

FIGS. 4A-4C illustrate an exemplary permeation process for modifying the permeability of the membrane of the biological cell membrane using the system.

FIGS. 5A-5C illustrate an exemplary poration process for creating a pore within the membrane of the biological cell using the system.

FIG. 6 is a flowchart that illustrates an exemplary process for modifying the biological cell.

### DETAILED DESCRIPTION OF THE INVENTION

An ultra-short pulsed laser may provide a capability to modify a biological cell. A modification to the biological cell may promote a transfection of the biological cell in accordance with various embodiments. The ultra-short pulsed laser may be fabricated using techniques of laser fabrication known in the art. In exemplary embodiments, the ultra-short pulsed laser emits optical pulses having temporal lengths in a range of picoseconds to femtoseconds (i.e., ultra-short) resulting in a very high electric field for an ultra-short duration. The optical pulses emitted from the ultra-short pulsed laser may be referred to as ultra-short pulses. Due to the ultra-short duration of the ultra-short pulses, as one skilled in the art will appreciate, processes involving the ultra-short pulses may be essentially athermal, resulting in a minimal transfer of heat energy. Furthermore, the processes involving the ultra-short pulses may be localized within various materials by focusing the ultra-short pulses, as described further herein.

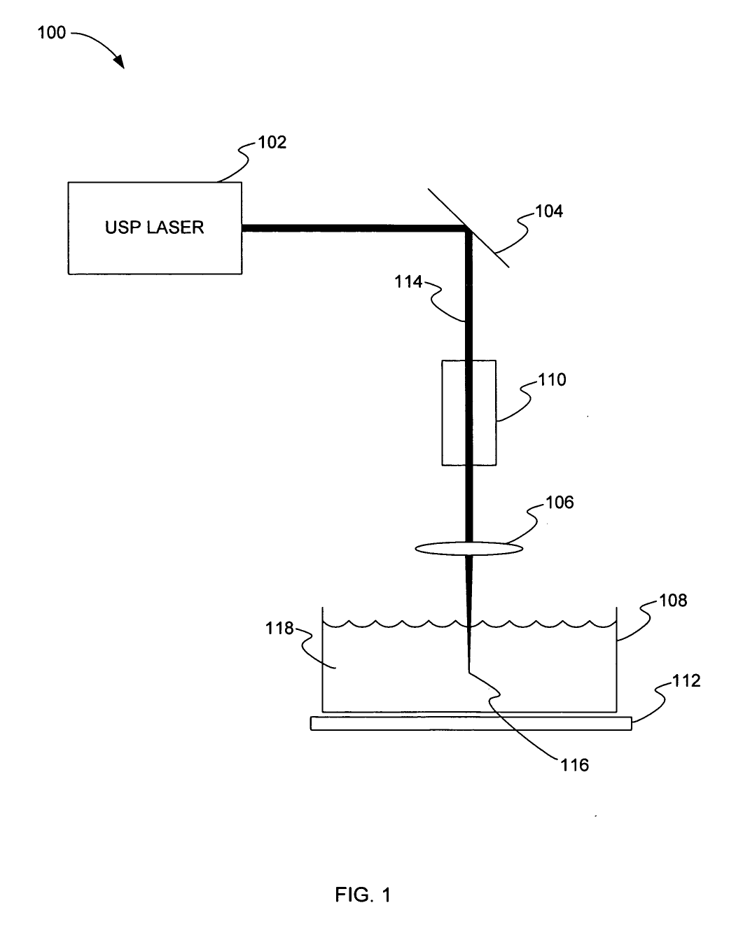

FIG. 1 illustrates an exemplary system 100 for modifying the biological cell. The system 100 may modify the biological cell in vitro and in vivo in various embodiments. The system 100 includes an ultra-short pulsed (USP) laser 102, a routing component 104, a focusing component 106, and a container 108. As will be apparent to those skilled in the art, the system 100 may optionally include a beam steerer 110 and/or a positioning stage 112. Although FIG. 1 describes the system 100 as including various constituent parts and components, fewer or more parts and components and/or arrangements of the parts and components may comprise the system 100 and still fall within the scope of various embodiments.

In exemplary embodiments, the ultra-short pulsed laser 102 emits a beam 114 comprising the ultra-short pulses. The routing component 104 may facilitate routing and/or directing of the beam 114 within the system. In some embodiments, the routing component 104 may include an optical fiber, or other waveguide, to which the beam 114 is coupled to. According to other embodiments, the routing component 104 may comprise conventional optical elements, such as mirrors and prisms, to direct and/or route the beam 114. Still other embodiments may include both the optical fiber and the conventional optical elements.

In various embodiments, the focusing component 106 may be configured to focus the beam 114 to form a focal zone 116, as described further herein. In one embodiment, the focusing component 106 may be affixed to the optical fiber. In some embodiments, the focusing component 106 may include a conventional lens. Some examples of the focusing component may include a compound lens. The compound lens may comprise multiple lenses in various configurations (e.g., Taylor-Cook Triplet, Zeiss Tessar, Orthoscopic Doublet, Zeiss Orthometer, Double Gauss, and Petzval). In one embodiment, the focusing component 106 may include a reflective focusing element (e.g., a parabolic mirror) configured to focus the beam 114 and form the focal zone 116.

The focusing component 106 may further include a beam splitter to create multiple beams of the ultra-short pulses in accordance with some embodiments. The beam splitter may comprise, for example, a fused fiber-based coupler, a beam splitter cube, and/or a series of beam splitters. Each of the multiple beams may correspond to a separate focusing component (e.g., the focusing component 106) to form multiple focal zones (e.g., the focal zone 116).

The container 108 may be configured to hold a mixture 118. According to various embodiments, the mixture 118 may comprise the biological cell and a medium. The medium is described further herein. The container may be replaced by a living organism, for example, in various in vivo applications of some embodiments (e.g., the living organism may comprise the mixture 118).

As mentioned herein, the system 100 may optionally include the beam steerer 110 and/or the positioning stage 112. In some embodiments, the beam steerer 110 and/or the positioning stage 112 may be configured to move the focal zone 116 relative to the mixture 118 held by the container 108. In one example, the positioning stage 112 may move the container 108 while the focal zone 116 is essentially stationary. In another example, the positioning stage 112 may move the optical fiber to which the beam 114 is coupled to, thereby moving the focal zone 116 relative to the container 108. Some embodiments of the system 100 may include more than one beam steerers and/or positioning stages (e.g., the beam steerer 110 and/or the positioning stage 112, respectively). The focal zone 116 may, for example, be moved in a raster pattern or to target a specific area within the mixture 118.

Some embodiments of the system 100 may include a stirring apparatus (not shown) configured to circulate the mixture 118 within the container 108. The stirring apparatus may, for example, include a magnetic stirrer, a gear driven motorized stirrer, or any other stirring means apparent to those skilled in the art. Additional components, such as a temperature regulation apparatus or any other regulatory, measurement, inspection, and/or analysis equipment, may be included in various embodiments.

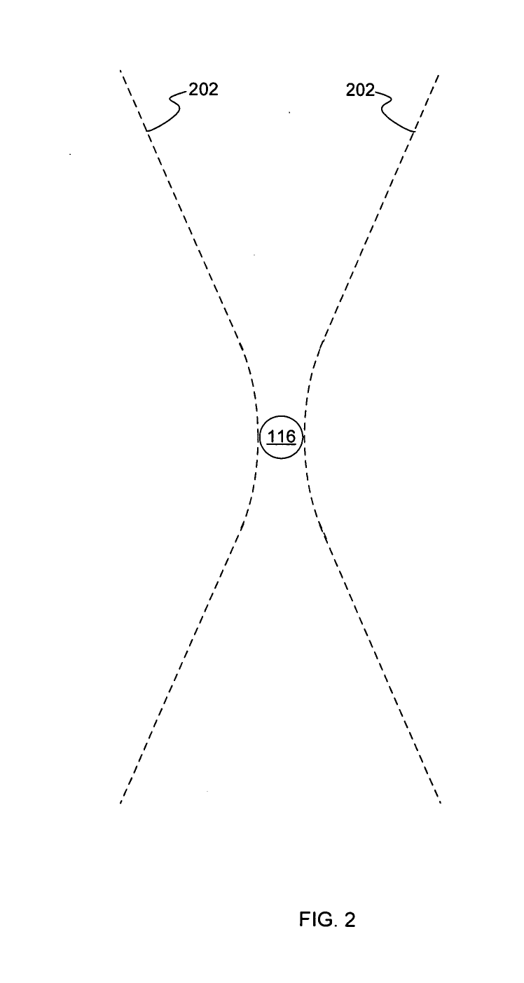

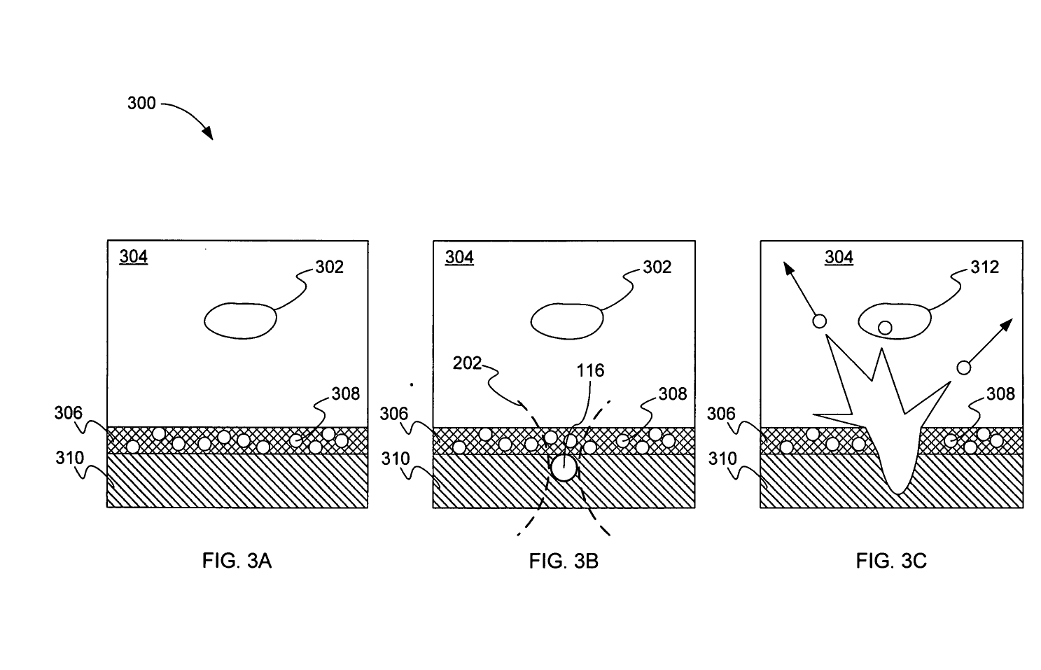

FIG. 2 illustrates the focal zone 116, according to exemplary embodiments. As mentioned herein, the focal zone 116 may be formed by focusing the beam 114 using the focusing component 106. Boundaries 202 may define the periphery of the beam 114 focused by the focusing component 106 near the focal zone 116. The focal zone 116 may be moved and/or positioned within various materials, including the mixture 118, using the positioning stage 112 and/or the beam steerer 110. The beam 114 comprising the ultra-short pulses may have numerous effects on the various materials at the focal zone 116, as described further herein and in connection with FIGS. 3A-6. Furthermore, operating conditions of the ultra-short pulsed laser 102, such as wavelength, pulse-rate, and/or output power, may be tuned to provide increased control of effects and processes occurring at the focal zone 116. As one skilled in the art will recognize, various materials away from the focal zone 116 may not be affected by the beam 114, thus providing, for example, localization of the effects and the processes occurring at the focal zone 116.

FIGS. 3A-3C illustrate an exemplary biolistic process 300 for transfecting the biological cell using the system 100. The term “biolistic” is a contraction of “biological” and “ballistic,” and is recognized in the art.

FIG. 3A depicts a biological cell 302, a medium 304, a dispersion material 306, and transfection agents 308. The biolistic process 300 may optionally include a rigid material 310. The biological cell 302 and the medium 304 may comprise a mixture (e.g., the mixture 118). The biological cell 302 may include any living biological cell or once living biological cell. According to some embodiments, the medium 304 may include an aqueous solution or other liquid. In other embodiments, the medium 304 may comprise a growth or culture medium designed to support growth of the biological cell 302.

The dispersion material 306 may include a plurality of transfection agents, such as the transfection agents 308. According to various embodiments, the dispersion material 306 may be heterogeneous or homogeneous. In examples where the dispersion material 306 is heterogeneous, the transfection agents 308 may include objects smaller than the biological cell 302 that are coated by, or otherwise associated with, the foreign objects and/or the substances (e.g., genes, DNA, RNA, drugs, and/or medicines) to be introduced into the biological cell 302. In one embodiment, the transfection agent may comprise heavy metal particles (e.g., gold or tungsten particles) that may be coated by foreign objects and/or the substances. In examples where the dispersion material 306 is homogeneous, the dispersion material 306 and the transfection agents 308 may be one and the same. The dispersion material 306 may be solid or semisolid. Additionally, some embodiments of the biolistic process 300 may not include the dispersion material 306, in which case the transfection agents 308 may be arranged on a surface of the rigid material 310 adjacent to the medium 304.

As mentioned herein, the biolistic process 300 may optionally include the rigid material 310. In one example, the rigid material 310 may support the dispersion material 306 in the medium 304. The rigid material 310 may also be a probe inserted in the medium 304 or a part of the container 108, in accordance with some embodiments.

FIG. 3B depicts the focal zone 116 positioned within the rigid material 310 using the system 100. As one skilled in the art will appreciate, when a level of energy delivered to the focal zone 116 by the beam 114 exceeds an ablation threshold of a material (e.g., the rigid material 310) in which the focal zone 116 is located, an explosive ablation event may be generated. The explosive ablation event may accelerate the transfection agents 308 proximate to the focal zone 116 in the medium 304. A path length in the medium 304 of the transfection agents 308 may depend, in part, on certain conditions of the biolistic process 300, such as intensity of the explosive ablation event, mass of the transfection agents 308, and viscosity of the medium 304

FIG. 3C depicts an aftermath of the explosive ablation event. As depicted, one of the transfection agents 308 accelerated in the medium 304 penetrates the cell 302 resulting in a transfected biological cell 312. Although only one of the transfection agents 308 is shown within the cell 302 in FIG. 3C, those skilled in the art will recognize that any number of the transfection agents 308 may penetrate the cell 302. The foreign objects and/or the substances associated with the transfection agents 308 contained by the transfected biological cell 312 may be released or dissociated from that transfection agent 308 into the transfected biological cell 312.

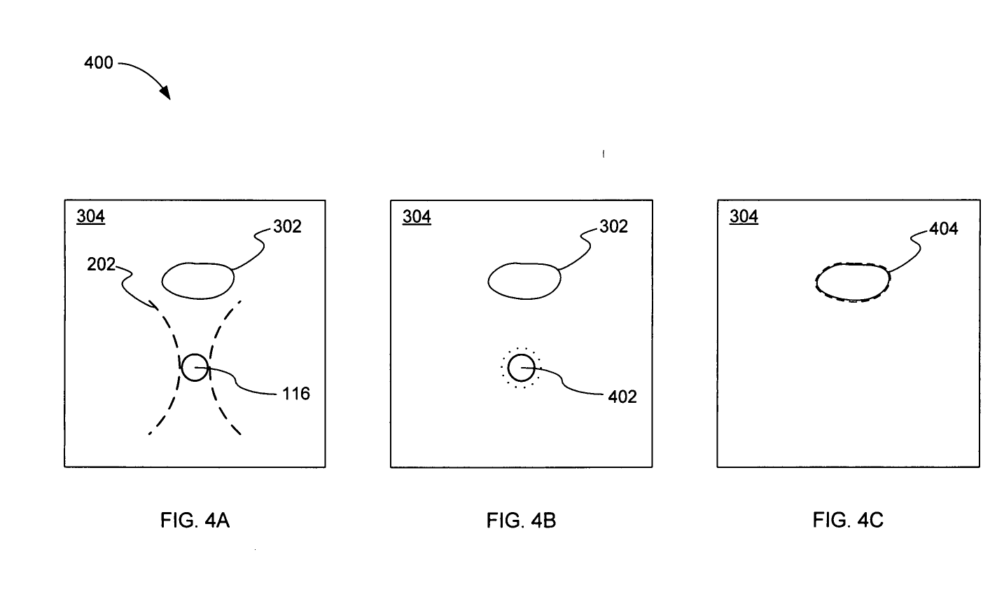

FIGS. 4A-4C illustrate an exemplary permeation process 400 for modifying the permeability of the membrane of the biological cell 302 membrane using the system 100. FIG. 4A depicts the biological cell 302, the medium 304, and the focal zone 116, which were described in connection with FIGS. 3A-3C. In exemplary embodiments, presence of the focal zone 116 within the medium 304 may result in a cavitation event. The cavitation event may induce a change in permeability of the membrane of the biological cell 302, thereby promoting transfection, for example. The cavitation event involved in the permeation process 400 may be described as the formation of a vapor bubble (also referred to as a cavitation bubble) within the medium 304 where a pressure falls below a vapor pressure of the medium 304 as a result of energy delivered to the focal zone 116 by the beam 114.

FIG. 4B depicts a cavitation bubble 402 formed due to vaporization of the medium 304 by energy delivered by the beam 114 at the focal zone 116. The cavitation bubble 402 may be proximate to the biological cell 302. In some instances, the cavitation bubble 402 may rapidly collapse, producing a shockwave in the medium 304. In other instances, the cavitation bubble 402 may be forced to oscillate in size or shape, producing periodic shock waves in the medium 304. Characteristics of the cavitation bubble 402, and the resulting shock waves, may be controlled by the operating conditions of the ultra-short pulsed laser 102 and how the system 100 is configured. For example, the cavitation bubble 402 may oscillate at a frequency related the pulse-rate of the ultra-short pulsed laser 102.

FIG. 4C depicts a permeated biological cell 404 modified by the shockwave that resulted from a rapid collapse of the cavitation bubble 402. According to various embodiments, specific mechanics involved in producing the permeated biological cell 404 in the permeation process 400 may be similar to that of sonoporation with an exception that the permeation process 400 is localized near the focal zone 116. Permeation of the biological cell 302 yielding the permeated biological cell 404 may promote transfection, for example, when the foreign objects and/or the substances are included in the medium 304 and adjacent to the permeated biological cell 404.

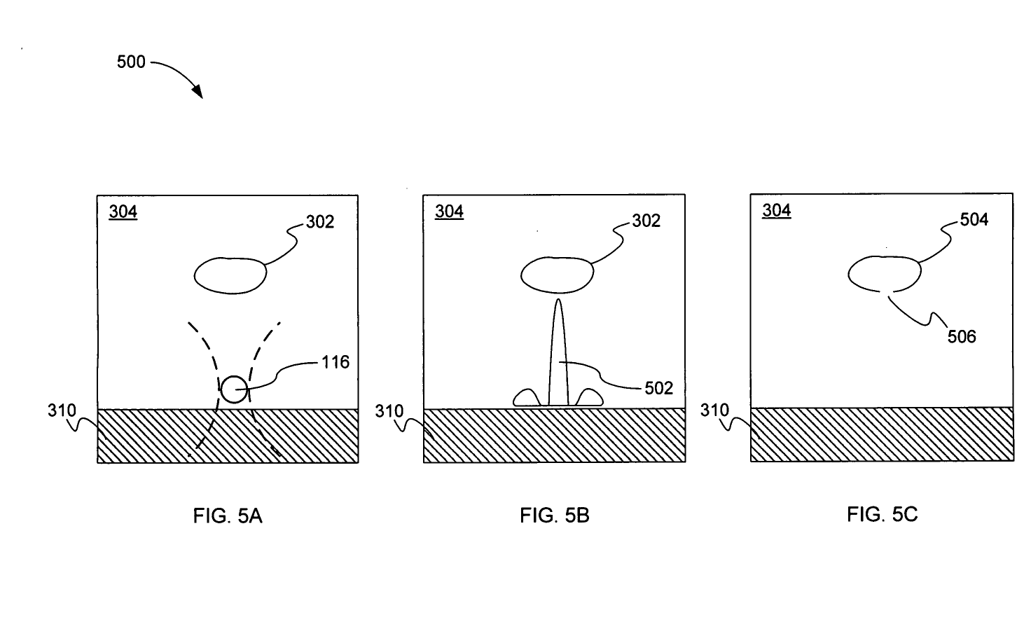

FIGS. 5A-5C illustrate an exemplary poration process 500 for creating a pore within the membrane of the biological cell 302 using the system 100. FIG. 5A depicts the biological cell 302, the medium 304, the focal zone 116, and the rigid material 310, which were described in connection with FIGS. 3A-3C. Similarly as described in connection with FIGS. 4A-4C, the presence of the focal zone 116 within the medium 304 may result in another cavitation event. However, due to proximity of the focal zone 116 to a surface of the rigid material 310 adjacent to the medium 304, the rapid collapse of another cavitation bubble may cause a high-speed jet (also referred to as a hydrojet) to be generated in the medium 304.

FIG. 5B depicts a hydrojet 502 formed as a result of the rapid collapse of the another cavitation bubble near the surface of the rigid material 310 adjacent to the medium 304. Similar to the cavitation bubble 402, characteristics of the hydrojet 502 may be controlled by the operating conditions of the ultra-short pulsed laser 102 and how the system 100 is configured. The characteristics may include flow-rate and dimensions of the hydrojet 502.

FIG. 5C depicts a poriferous biological cell 504 having a pore 506 created by the hydrojet 502 puncturing the membrane of the biological cell 302. According to various embodiments, the pore 506 may be transient or static. To illustrate, the pore 506 may close at some point in time subsequent to creation of the pore 506. In another example, the foreign objects and/or the substances, which may be included in the medium 304, may be forced into the biological cell 302 by the hydrojet 502.

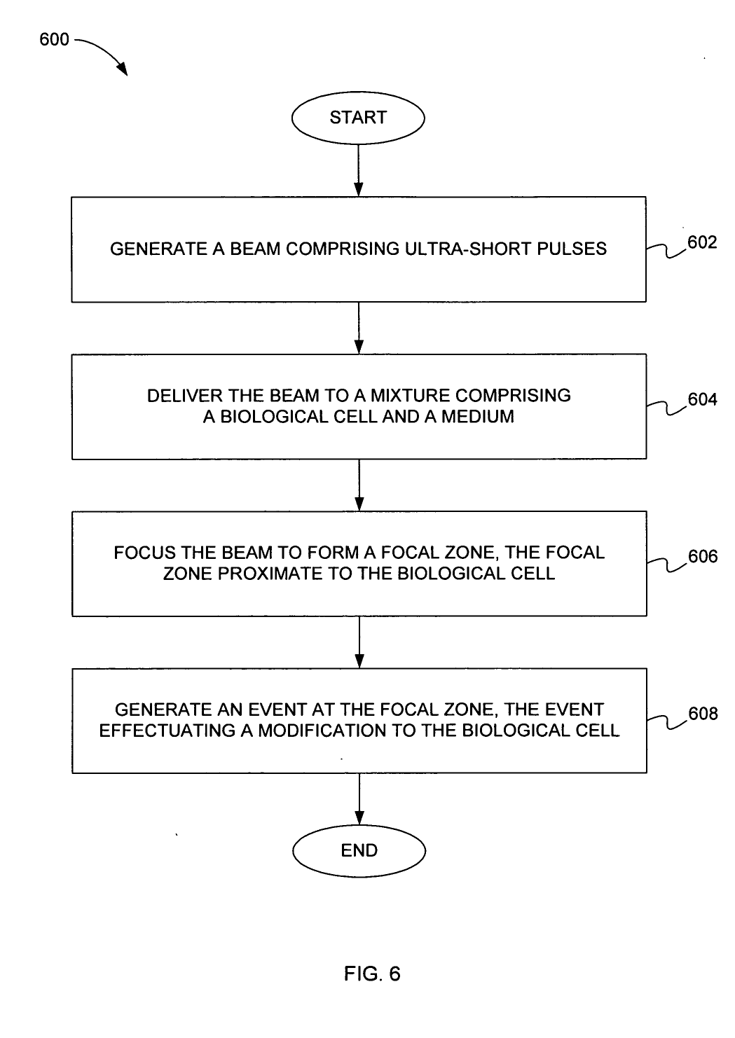

FIG. 6 is a flowchart 600 that illustrates an exemplary process for modifying a biological cell, such as the biological cell 302 depicted in FIGS. 3A-5C. According to various embodiments, the process may be carried out using the system 100 to provide numerous modifications (e.g., transfection, permeation, and/or poration) to the biological cell.

At step 602, a beam (e.g., the beam 114) comprising ultra-short pulses is generated. In some embodiments, the beam 114 may be generated, for example, by the ultra-short pulsed laser 102. In other embodiments, the beam 114 may be generated by any light source capable of generating the ultra-short pulses. The light source capable of generating the ultra-short pulses may include fiber mode-locked lasers, gas lasers (e.g., helium-neon, argon, and krypton), chemical lasers (e.g., hydrogen fluoride and deuterium fluoride), dye lasers, metal vapor lasers (e.g., helium cadmium metal vapor), solid state lasers (e.g., titanium sapphire and neodymium yttrium aluminum garnet), or semiconductor lasers (e.g., gallium nitride and aluminum gallium arsenide), for example.

At step 604, the beam 114 is delivered to a mixture comprising a biological cell and a medium (e.g., the mixture 118). According to various embodiments, the beam 114 may be delivered by the routing component 104. In one example, the routing component 104 may include an optical fiber, or other waveguide, to which the beam 114 is coupled to. In another example, the routing component 104 may comprise conventional optical elements, such as mirrors and prisms, to direct and/or route the beam 114. Additionally, as mentioned herein, the mixture 118 may be held by a container (e.g., the container 108). However, the living organism may comprise the mixture 118 in accordance with some embodiments.

At step 606, the beam 114 is focused to form a focal zone (e.g., the focal zone 116), whereby the focal zone 116 is proximate to the biological cell. The focal zone 116 is further described in connection with FIG. 2. In some embodiments, the beam 114 may be focused by the focusing component 106. As mentioned herein, the focusing component 106 may, for example, be affixed to the optical fiber to which the beam 114 is coupled to. The focusing component 106 may include a conventional lens and/or a compound lens, according to various embodiments. According to one embodiment, step 606 may further include splitting the beam 114 to create multiple beams of the ultra-short pulses. Each of the multiple beams may, for example, be focused to form multiple focal zones.

At step 608, an event is generated at the focal zone 116 that effectuates a modification to the biological cell 302. As discussed herein, various events may be generated at the focal zone 116, which may bring about various modifications to the biological cell 302.

In various embodiments, the event generated at step 608 may include the explosive ablation event similar to that described in connection with FIGS. 3A-3C. As discussed herein, the explosive ablation event may be generated when the level of energy delivered to the focal zone 116 by the beam 114 exceeds the ablation threshold of the material in which the focal zone 116 is located. The explosive ablation event may cause the biological cell 302 to be modified. For instance, if the explosive ablation event occurs at or near some material containing projectiles, then the projectiles may be propelled or accelerated in the mixture 118. A material containing projectiles may comprise the dispersion material 306. The projectiles may include the foreign objects or the substances associated with relatively massive particles (e.g., the transfection agents 308). While passing through the mixture 118, the projectiles may impinge on the biological cell 302. Given sufficient momentum, the projectiles may penetrate the membrane of the biological cell 302, thus leading to transfection.

According to some embodiments, the event generated at the step 608 may include various cavitation events similar to those described in connection with FIGS. 4A-4C and 5A-5C. As discussed herein, the cavitation event may include the formation of a cavitation bubble at the focal zone 116, such as the cavitation bubble 402. Proximity of the biological cell 302 to the cavitation bubble 402 may cause the biological cell 302 to be modified. To illustrate, the rapid collapse of the cavitation bubble 402 may produce a shockwave in the medium 304. Subjection to the shockwave may, for example, alter the permeability of the membrane of the biological cell 302 resulting in the permeated biological cell 404. Furthermore, the cavitation bubble 402 may be forced to oscillate at a frequency related to certain beam characteristics (e.g., the pulse rate) resulting in periodic shockwaves, as mentioned herein. The membrane of the permeated biological cell 304 may be, for example, more susceptible to the introduction of the foreign objects and/or substances present in the medium 304.

In another embodiment, the event generated at the step 608 may include a cavitation event occurring near a surface of a rigid material (e.g., the surface of the rigid material 310) at the focal zone 116. As one skilled in the art will recognize and as discussed herein, a hydrojet, such as the hydrojet 502, may be generated as a result of the rapid collapse of a cavitation bubble near the surface of the rigid material 310. As the hydrojet 502 extends into the medium 304, the hydrojet 502 may puncture the membrane of the biological cell 302 leaving a pore in the membrane. The foreign objects and/or the substances may be readily introduced to a poriferous cell, such as the poriferous cell 504, having the pore 506 created by the hydrojet 502.

The above description is illustrative and not restrictive. Many variations of the invention will become apparent to those of skill in the art upon review of this disclosure. The scope of the invention should, therefore, be determined not with reference to the above description, but instead should be determined with reference to the appended claims along with their full scope of equivalents.

What is claimed is:

1. A method for modifying a biological cell comprising:
generating a beam comprising ultra-short pulses;
delivering the beam to a mixture comprising a biological cell and a medium;
focusing the beam to form a focal zone, the focal zone proximate to the biological cell; and
generating an event at the focal zone, the event effectuating a modification to the biological cell.

2. The method of claim 1, further comprising positioning a material containing a transfection agent adjacent to the mixture.

3. The method of claim 2, wherein generating the event comprises generating an explosive ablation event, the explosive ablation event propelling the transfection agent in the mixture.

4. The method of claim 2, wherein the transfection agent includes a genetic material.

5. The method of claim 2, wherein the transfection agent includes a pharmaceutical material.

6. The method of claim 1, wherein generating the event comprises generating a cavitation event.

7. The method of claim 6, wherein the cavitation event produces a shockwave in the mixture.

8. The method of claim 6, wherein the cavitation event occurs near a rigid surface effectuating a hydrojet in the mixture, the rigid surface located adjacent to the mixture.

9. The method of claim 1, wherein the modification includes a pore in a membrane of the biological cell.

10. The method of claim 1, wherein the modification includes an alteration of a permeability of a membrane of the biological cell.

11. The method of claim 1, further comprising circulating the mixture.

12. The method of claim 1, further comprising moving the focal zone within the mixture.

13. A system for modifying a biological cell comprising:
a laser configured to generate a beam comprising ultra-short pulses;
a routing component configured to deliver the beam to a container holding a mixture, the mixture comprising a biological cell and a medium; and
a focusing component configured to focus the beam to produce a focal zone proximate to the biological cell, the focal zone generating an event.

14. The system of claim 13, wherein the routing component comprises an optical fiber.

15. The system of claim 13, further comprising a stirring apparatus configured to circulate the mixture within the container.

16. The system of claim 13, further comprising a positioning stage configured to move the mixture relative to the focal zone.

17. The system of claim 16, further comprising a plurality of positioning stages.

18. The system of claim 13, further comprising a beam steerer configured to move the focal zone relative to the mixture.

19. The system of claim 18, further comprising a plurality of beam steerers.

20. The system of claim 13, wherein the container is a living organism.
